(19) United States
(12) Patent Application Publication
(10) Pub. No.: US 2011/0190251 A1
(43) Pub. Date: Aug. 4, 2011

(54) METHOD FOR INHIBITING THE BUILD-UP OF ARTERIAL PLAQUE BY ADMINISTERING FULLERENES

(76) Inventors: Christopher L. Kepley, Ringgold, VA (US); Robert P. Lenk, Danville, VA (US); Stephen R. Wilson, Danville, VA (US); Zhiguo Zhou, Winston-Salem, NC (US)

(21) Appl. No.: 12/921,083

(22) PCT Filed: Mar. 3, 2009
(86) PCT No.: PCT/US2009/001335

§ 371 Date: Dec. 16, 2010

Related U.S. Application Data
(60) Provisional application No. 61/033,336, filed on Mar. 3, 2008.

(51) Int. Cl. A61K 31/216; C07C 69/743; A61K 31/575; A61P 9/00

(52) U.S. Cl. 514/182; 514/510; 560/80

(57) ABSTRACT

Disclosed herein are methods of inhibiting the build-up of arterial plaque in a subject in need thereof. These methods comprise administering to the subject in need thereof a therapeutically effective amount of fullerenes.

### BACKGROUND

The present disclosure relates to a method for inhibiting the build-up of arterial plaque in humans.

Cardiovascular disease is a major health risk throughout the industrialized world. Atherosclerosis, the most prevalent of cardiovascular diseases, is the principal cause of heart attack, stroke, and gangrene of the extremities, and thereby the principal cause of death in the United States. Atherosclerosis is a complex disease involving many cell types and molecular factors (for a detailed review, see Ross, Nature 362:801-809 (1993)).

Atherosclerosis is a disease characterized by the deposition of fatty substances, primarily cholesterol, and subsequent fibrosis in the inner layer (intima) of an artery, resulting in plaque deposition on the inner surface of the arterial wall and degenerative changes within it. The ubiquitous arterial fatty plaque is the earliest lesion of atherosclerosis and is a grossly flat, lipid-rich atheroma consisting of macrophages (white blood cells) and smooth muscle fibers. The fibrous plaque of the various forms of advanced atherosclerosis has increased intimal smooth muscle cells surrounded by a connective tissue matrix and variable amounts of intracellular and extracellular lipid. At the luminal surface of the artery, a dense fibrous cap of smooth muscle or connective tissue usually covers this plaque or lesion. Beneath the fibrous cap, the lesions are highly cellular consisting of macrophages, other leukocytes and smooth muscle cells. Deep in this cell-rich region may be areas of cholesterol crystals, necrotic debris and calcification.

If allowed to progress, the disease can cause narrowing and obstruction of the lumen of the artery, diminished or occluded blood flow and, consequently, ischemia or infarction of the predominantly affected organ or anatomical part such as the brain, heart, intestine or extremities. The result can be significant loss of function, loss of cellular substance, emergency medical and/or surgical procedures, and significant disability or death. Alternatively, the arterial wall can be severely weakened by the infiltration of the muscular layer with the lipid (cholesterol), inflammatory white blood cells, connective tissue and calcium, resulting in soft and/or brittle areas which can become segmentally dilated (aneurysmal) and rupture or crack leading to organ, limb or even life-threatening hemorrhage.

Once the disease has progressed to the stage of significant persistent symptoms and compromised function, the next treatment step has conventionally been artery bypass grafting to repair and/or replace the damaged artery. While coronary artery bypass has become one of the more common major cardiovascular surgical procedures in the United States, surgery clearly is not the solution to the pathologic process. Moreover, there is a significant risk of morbidity and mortality associated with surgery that many patients are reluctant to accept. Indeed, the autogenous veins or arteries used to bypass the disease-impaired arteries undergo atherosclerosis changes postoperatively generally at a faster rate than the original, affected arteries. The Coronary-Artery Surgery Study (CASS) sponsored by the National Heart, Lung and Blood Institute (NHLBI) concluded that certain subsets of patients do not gain any overall statistical benefit from bypass surgery in comparison to other medical treatments. Carraciolo, Circulation, 91(9): 2335-44 (1995).

As an alternative to coronary bypass surgery, certain medications and procedures are used to treat the results of atherosclerosis. These treatments include chelation with ethylene diamine tetra-acetic acid (EDTA) and percutaneous transluminal coronary angioplasty (PTCA). EDTA treatments, however, are still experimental, unproved and potentially as harmful as they are beneficial. PTCA treatments are invasive, of limited application and success and occasionally manifest lethal complications. Highly experimental intra-arterial laser beam plaque vaporization has limited application and requires an open operative approach to affected vessels.

It is now well established that vascular blockage and cardiovascular disorders including myocardial infarction, coronary heart disease, hypertension and hypotension, cerebrovascular disorders including stroke, cerebral thrombosis and memory loss due to stroke; peripheral vascular disease and intestinal infarction are caused by blockage of arteries and arterioles by atherosclerotic plaque. The production of atherosclerotic plaque formation is multi-factorial in its production. Hypercholesterolemia, especially elevated levels of low-density lipoprotein cholesterol (LDL) is an important risk factor for atherosclerosis and arteriosclerosis and associated diseases.

Lipoproteins are spherical particles with the lipophilic triglycerides and cholesteryl esters in the hydrophobic core, and the amphiphilic lipids, phospholipids and free cholesterol on the surface with apolipoproteins. When the amount of cholesterol entering the body increases, the pools of sterol within liver cells expands and the receptors that clear LDL from the blood down-regulate, thus increasing LDL levels in the blood. When cholesterol intake is constant, some long-chain saturated fatty acids further suppress the hepatic LDL receptor whereas several unsaturated fatty acids have the opposite effect. Lipoprotein (a) [Lp (a)] has emerged as a plasma lipoprotein linked to both diseases of the coronary arteries, the carotid and the cerebral arteries. It is structurally related to LDL and possesses one molecule of apolipoprotein B₁₀₀ per particle. Macrophages express the scavenger receptor that readily recognizes oxidatively modified Lp (a). Marcovina & Morrisett, Current Opinion In Lipidology, 6:136-145 (1995).

Cholesterol levels below 200 mg/dl are considered “desirable.” A Scandinavian study showed that reduction of cholesterol reduced mortality associated with coronary artery disease (CAD) by 42% over six year period and reduced overall mortality by 30%. J. Hardman & L. Lipman, Goodman & Gilman's The Pharmacological Basis Of Therapeutics (9th ed. 1996) (hereinafter “J. Hardman”). Researchers have shown that a 1-mMol/L increase in triglyceride levels produces a 76% increase in cardiovascular disease risk in women and a 31% increase in men. Austin, American Journal of Cardiology, 83 (9B):13F-16F (1999). Even in patients with established disease, lowering of LDL cholesterol to between 2 and 2.5 mmol/L retards its progression and may even lead to regression. Illingsworth, Drugs, 41(20):151-160 (1991).

It is recommended that persons with elevated cholesterol concentrations above 240 mg/dL (6.2 mM/L) receive treatment and that those with borderline values between 200-239 mg/dL (5.2 to 6.2 mM/L) be further evaluated according to the presence of risk factors for coronary artery disease including the sex of the patient, post-menopausal status, a low plasma concentration of high-density lipoproetin cholesterol (HDL) cholesterol (below 35 mg/dL [0.9 mM/L]), positive family history, smoking, hypertension and diabetes mellitus. Expert Panel on Detection, Evaluation, and Treatment of High Blood Cholesterol in Adults, J. Am. Medical A., 269(23):3015-3023 (1993). Other factors include obesity, hypertriglyceridemia, sedentary lifestyle, steroid use, .beta.-adrenergic blocking agents, some diuretics and genetic factors. Frohlich & Pritchard, Clinical Biochemistry, 22:417-433 (1989).

By the 1980's, it was recognized that HDL levels could be more important in predicting atherosclerotic disease than LDL and that HDL may prevent the development of CAD. Id. Factors such as smoking, obesity, hypertriglyceridemia, genetic factors and lack of exercise are major causes of reduced serum HDL. HDL cholesterol lipoproteins move excess cholesterol from the extrahepatic organs to the liver for excretion. Dietschy, Am. J. Clinical Nutrition, 65:1581 S-9S (1997). There is evidence that virtually every body tissue is capable of at least some cholesterol synthesis from the precursor acetyl-coenzyme A (CoA). Every day, HDL carries back to the liver an amount of cholesterol equal to the amount synthesized and taken up as LDL by all extrahepatic organs except endocrine glands. There is a second LDL transport process that is receptor independent. Id. Removal of free cholesterol from arterial wall cells may be an important mechanism by which HDL plays an anti-atherogenic role. J. Hardman, supra, at 878.

The earliest recognized gross lesion in atherogenesis is the fatty streak, characterized by an accumulation of cells loaded with cholesteryl esters (“foam cells”) just beneath the vascular endothelium. The LDL receptor in the arteries gives rise to foam cells and fatty streaks, the earliest lesion in atherosclerosis, but there is also a receptor-independent mechanism for their formation. This has been demonstrated by the development of lesions rich in macrophage-derived foam cells, even in patients and animals deficient in LDL receptors, and the failure to produce foam cells from normal monocytes and monocyte derived macrophages incubated with LDL. This led researchers to explore the possibility of a post-secretory modification of LDL before it is taken up into foam cells by a new, specific receptor: the “scavenger receptor.” Steinberg, New Eng J. Medicine, 320(14):915-924 (1989).

At any given level of hypercholesterolemia there is considerable variation in clinical disease. Postsecretory modifications in the structure of lipoproteins appear to affect their atherogenic potential. Steinberg, supra, at 915. It is not only the elevated levels of LDL cholesterol that are important, but also its oxidation that leads to atherosclerosis. For this reason antioxidants are believed to reduce the risk of atherosclerotic disease. Mortensen, Molecular Aspects of Medicine, 18:s137-s144, (Supp. 1997). Peroxidation of polyunsaturated fatty acids in the LDL lipids is the common initiating factor of the changes and the cytotoxicity of oxidized LDL has been proven by several research groups and may lead to the denudation of the benign fatty-streak lesion into the atheromatous plaque. Steinberg, supra, at 918.

Researchers believe that the oxidation of LDL within the arterial wall itself is most important. Ocana, New Eng. J. Medicine, 321(17):1196-1197 (1989). Auto-antibodies to MDL-LDL were seen at significantly higher titers in men with atherosclerosis than in normal controls, and in a greater proportion of smokers, those with higher LDL cholesterol, and those with higher serum levels of copper in the case group. Salonen, 339 LANCET 883-887 (1992).

Researchers also have studied the effects of incubation of LDL with macrophages and found that in that environment LDL is oxidized and recognized and taken up by the acetyl LDL or scavenger receptor in the same cell. Alpha-tocopherol, butylated hydroxytoluene (BHT) and Probucol block this process. Parthasarathy, Arteriosclerosis, 6(5):505-10 (1986). Treatment with Probucol, a potent anti-oxidant, significantly lowered the rate of development of fatty streak lesions in hyperlipidemic rabbits, although the plasma cholesterol level was not lower than in lovastatin-treated animals. Carew, Schwenke & Steinberg, PNAS USA, 84:7725-7729 (1987). Similar results have been demonstrated in cultures of LDL with endothelial cells. Steinbrecher, PNAS, 81:3883-3887 (1984). Monocytes and neutrophils, when incubated with LDL, oxidize LDL and render it toxic. Cathcart, Morel & Chisolm, J. Leukocyte Biology, 38:341-350 (1985).

Fullerenes are a family of carbon allotropes that comprise closed cages of generally 20 to 200 carbon atoms and may also include chemical moieties attached to the exterior or incorporated within the cage. Fullerenes can be in the form of a hollow sphere, ellipsoid, or tube. The most common fullerene to date is the C₆₀ Buckminsterfullerene (IUPAC name (C60-Ih)[5,6]fullerene). Another fairly common buckminsterfullerene is C₇₀, but fullerenes with 72, 76, 84 and even up to 200 carbon atoms can be obtained. Fullerenes can contain 500 or more carbon atoms.

Structural variations include nonclosed-cage structures, heterofullerenes, derivatives formed by substitution of hydrofullerenes, the fusion of organic rings or ring systems to the fullerene cage, chiral fullerenes, buckyball clusters, nanotubes, megatubes, polymers, nano “onions,” linked “ball-and-chain” dimers, and fullerene rings. See, e.g., Miessler and Tarr, Inorg. Chem. 3, Pearson Education International. ISBN 0-13-120198-0 (2004); Mitchel et. al., Inorg. Chem., 40: 2751 (2001); Sano, Nature (London), 414: 506 (2001); Shvartsburg, Phys. Chem. 103: 5275 (1999); and Li et al., Chem. Phys. Lett. 335: 524 (2001).

In general, fullerenes are hydrophobic and sparingly soluble in many solvents. See, e.g., Braun et al., Fullerenes, Nanotubes and Carbon Nanostructures, 15; 311-314 (2007). However, a variety of procedures for functionalizing fullerenes are known in the art, and some of the derivative fullerenes are water soluble. See, e.g., U.S. Pat. No. 5,648,243 to Chiang; U.S. Patent Application Publication Nos. 2008/0004345 and 2004/0044062; Jensen et al., Bioorganic & Medicinal Chemistry, 4:767-79 (1996); Da Ros et al., Croatica Chemica Acta CCACAA 74:743-55 (2001); Wilson, “Perspectives in Fullerene Nanotechnology,” Osawa, ed., (Kluwer Academic Publishers, Dorcrecht, Netherlands, 2000); Syrensky, et al., Kopf Carrier #63, (David Kopf Instruments Tujunga, Calif., September 2006); Y. L. Lai and L. Y. Chiang, J. Autonomic Pharmacol., 17:229 (1997); Schinazi et al., Proc. Electrochem. Soc., 97:10, (1997); Lai et al., World J. Surg., 24:450 (2000); Jin et al., J. Neuroscience Res., 62:600 (2000); Huang et al., Free Radical Biol. Med., 30:643 (2001); Chi et al., “Perspectives of Fullerene Nanotechnology,” pp 165-183, E. Osawa ed., (Kluwer Academic Publisher, Great Britain, 2002); Dugan et al., P.N.A.S. 94:9434-39 (1997); Dugan et al., Parkinsonism & Related Disorders 7:243-46 (2001); Quick et al., Neurobiol of Aging (electronic publication 2006); Kato et al., Chem & Biodiv., 2:1232-1241 (2005); Georgakilas et al, Proc. Nat. Acad. Sci. 99; 5075-5080 (2002).

Incorporation of fullerenes into lipid vesicles has also been studied (see, e.g., Bensasson et al., Journal of Physical Chemistry, 98:3492-3500 (1994); Hirsch et al., Angewandte Chemie International Edition, 39:1845-1848 (1999); U.S. Pat. No. 7,070,810; Felder, et al., Helv. Chim. Acta, 85: 288-319 (2002).

Fullerenes can also be modified at their surface to present specific biologically active groups, such as lectins or antibodies. See, e.g., U.S. Patent Application Publication No. 2005/0043787; U.S. Pat. No. 5,310,669. Certain chemically modified fullerenes are commercially available. See, e.g., BuckyUSA, Houston, Tex. and American Dye Source, Inc., Quebec, Canada.

Fullerenes and derivatives of fullerenes have been proposed as free radical scavengers. See, e.g., Haddon, J. Am. Chem. Soc. 112:3389 (1990); U.S. Pat. No. 5,648,243 to Chiang, U.S. Patent Application Publication No. 2003/0162837 by Dugan; U.S. Pat. No. 7,163,956 to Wilson; Kepley, J. Immunol. 179:665 (2007).

### SUMMARY

Disclosed herein are methods of inhibiting the build-up of arterial plaque in an individual.

According to various embodiments, disclosed herein are methods of inhibiting the build-up of arterial plaque in a subject in need thereof. These methods comprise administering to the subject in need thereof a therapeutically effective amount of fullerenes.

### BRIEF DESCRIPTION OF THE DRAWINGS

FIG. 1 illustrates non-limiting examples of synthetically modified fullerenes with any combination of hydrophilic, lipophilic, or amphiphilic chemical moieties.

FIG. 2 illustrates additional non-limiting examples of synthetically modified fullerenes with any combination of hydrophilic, lipophilic, or amphiphilic chemical moieties.

FIG. 3 illustrates additional non-limiting examples of synthetically modified fullerenes with any combination of hydrophilic, lipophilic, or amphiphilic chemical moieties.

FIG. 4 illustrates non-limiting examples of water soluble and water insoluble fullerenes.

### DETAILED DESCRIPTION

In accordance with this detailed description, the following abbreviations and definitions apply. It must be noted that as used herein, the singular forms “a”, “an”, and “the” include plural referents unless the context clearly dictates otherwise. Thus, for example, reference to “compounds” includes a plurality of such compounds and reference to “the dosage” includes reference to one or more dosages and equivalents thereof known to those skilled in the art, and so forth.

The publications discussed in this disclosure are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed.

Unless otherwise stated, the following terms used in the specification and claims have the meanings given below:

“Fullerene” or “fullerene molecule” as used herein refers to any member of the fullerene family of carbon cage molecules. Fullerenes are generally carbon structures formed of five and six membered rings arranged so that the rings form a closed geodesic sphere or spheroid held together by a combination of single and double carbon:carbon covalent bonds. The fullerenes in this disclosure can be defined by the formula: C₂, wherein s is greater than or equal to 30, such as from about 30 to about 200 or from about 30 to about 100. For example, the fullerenes include C₆₀, C₇₀, and similar molecules that range in molecular weight from C₆₀ up to C₈₄, C₉₀, and larger such molecules, with shapes ranging from spheroidal to ellipsoidal, elongated and other shapes, and including not only single-walled but also multi-walled cages consisting of stacked or parallel layers. The fullerenes may be unmodified or underivatized. Alternatively, the fullerenes may enclose one or more atoms such as metal atoms, or other small chemical groups, inside the carbon cage; such fullerenes are sometimes called endohedral fullerenes. Fullerenes, as used herein, also includes structures with chemical functional groups attached to the surface of the carbon cage. The functional groups can be covalently bound to the carbon cage via opening carbon:carbon double bonds. Fullerenes also include other structural variants, derivatives, and/or modified or functionalized fullerenes as described herein and/or as known in the art. The fullerenes can be synthetic or naturally-occurring. Synthetic fullerene molecules can be prepared in a laboratory by known methods (see, e.g., U.S. Pat. No. 5,177,248 and Krätschmer et al., Chem. Phys. Lett., 170, 167-170 (1990)) or can be purchased commercially.

In one embodiment, the fullerenes are water soluble, meaning the fullerenes distribute more or less uniformly in an aqueous solution and do not significantly precipitate. Water soluble fullerenes are known in the art as described above, and can be synthesized for example by attaching one or more hydrophilic chemical groups to the surface of the carbon cage. Suitable hydrophilic chemical groups include hydroxyl or polyhydroxyl groups and N-ethylpolyamino groups. Non-limiting examples of water soluble fullerenes include C₆₀(OH)ₙ, C₆₀(NH—CH₂—CH₃)ₙ, and C₇₀-Tetraglycolate. Many other examples of water-soluble fullerenes are known and can involve the addition of one or more charged groups such as phosphates, sulfates, ammonium, carboxylates, or other charged groups; or hydrophilic, such as hydroxyl and polyhydroxyl groups; and carbohydrates, peptides, proteins, nucleotides and DNA.

In another embodiment, chemical groups such as amphiphilic or lipophilic groups can be attached to the carbon cage instead of or in combination with hydrophilic chemical groups.

“Fullerene” or “fullerene molecule” as used herein refers to certain synthetically modified fullerene molecules as described herein, including amphiphilic or lipophilic synthetically modified fullerenes of the formula Zₘ-F-Yₙ; and hydrophilic or amphiphilic synthetically modified fullerenes of the formula Z′ₘ-F-Y′ₙ. The fullerenes comprise closed cages of 60 to 200 carbon atoms which may also include chemical moieties attached to the exterior and/or incorporated within the cage.

The amphiphilic or lipophilic synthetically modified fullerene molecules are described in copending U.S. patent application Ser. No. 02/073,230, U.S. Patent Application Publication No. 2008-0213324-A1, filed Mar. 3, 2008, entitled “AMPHIPHILIC OR LIPOPHILIC POLAR FUNCTIONALIZED FULLERENES AND THEIR USES,” the entire disclosure of which is incorporated by reference herein.

The amphiphilic or lipophilic and hydrophilic or amphiphilic synthetically modified fullerene molecules as described in the copending application include fullerenes that have an aspect ratio≠1, with an equatorial band and two opposing poles, and comprise an adduct at one or both poles.

In one embodiment, the amphiphilic or lipophilic synthetically modified fullerene has the formula

Zₘ-F-Yₙ;

wherein F is a fullerene of formula Cₚ or X@Cₚ, the fullerene having two opposing poles and an equatorial region;

Cₚ represents a fullerene cage having p carbon atoms, and X@Cₚ represents such a fullerene cage having a chemical group X within the cage.

Z and Y are positioned near respective opposite poles of Cₚ;

m=1-5 and Z is a hydrophilic, lipophilic, or amphiphilic chemical moiety;

n=1-5 and Y is a lipophilic chemical moiety;

p=60-200 and p is an even number; and

X, if present, represents one or more metal atoms within the fullerene (F), optionally in the form of a trinitride of formula Gᵢ₌₁₋₃Hₖ₌₃₋ᵢN in which G and H are metal atoms.

In exemplary variations p is an even number between 60 and 120, with p=60-96 being more common and p=60 or p=70 being preferred. The synthetically modified fullerene can be arranged wherein each chemical moiety Z is composed of formula AᵣB in which A is a hydrophilic, lipophilic or amphiphilic chemical moiety, r=1-4, and B is a chemical linker connecting said A to the fullerene, and each chemical moiety Y is composed of formula DE, in which E is a lipophilic chemical moiety, v=1-4, and D is a chemical linker connecting the lipophilic chemical moiety to the fullerene.

The amphiphilic or lipophilic synthetically modified fullerene can be a prolate ellipsoid shaped fullerene having a major axis such that said poles are located at opposing ends of the major axis of the prolate ellipsoid fullerene. Alternatively, the fullerene can be spheroid with opposing poles defined by an axis through opposing carbon rings. Z and Y can configured such that when the molecule is contacted with a lipid bilayer in an aqueous medium, the equatorial region of F is selectively located within or in close proximity to the phospholipid bilayer. The molecule can be configured so that in an extended configuration has an aspect ratio of about 2.1 to 15, and a diameter less than about 2 nm. Such configurations are preferred configurations for incorporation of the molecules into lipid bilayers.

In another embodiment, the amphiphilic or lipophilic synthetically modified fullerene molecule has the formula Z(Cₚ)Y wherein: p=60-200 carbons, preferably p=60 or 70; Y is a lipophilic moiety covalently connected to Cₚ, optionally through a linking group, at or near a pole thereof, and wherein Z is a lipophilic moiety, amphiphilic moiety, or a hydrophilic moiety covalently connected to Cₚ, optionally through a linking group, at or near a pole opposite to said Y; and,

wherein said lipophilic moiety Y is capable of anchoring the synthetic fullerene molecule to a lipid membrane;

In another embodiment, the amphiphilic or lipophilic synthetically modified fullerene molecule has the formula Z(Cₚ)Y wherein: p=60-200 carbons, preferably p=60 or 70; Y is a lipophilic moiety covalently connected to Cₚ, optionally through a linking group, at or near a pole thereof, and wherein Z is a hydrophilic moiety covalently connected to Cₚ, optionally through a linking group, at or near a pole opposite to said Y; and, wherein said lipophilic moiety Y is capable of anchoring the synthetic fullerene molecule to a lipid membrane.

In another embodiment, the amphiphilic or lipophilic synthetically modified fullerene molecule has the formula Z(C₇₀)Y; wherein Y is a lipophilic moiety covalently connected to C₇₀, optionally through a linking group, at or near a pole thereof, and wherein Z is a lipophilic moiety, amphiphilic moiety, or a hydrophilic moiety covalently connected to C₇₀, optionally through a linking group, at or near a pole opposite to said Y; and, wherein said lipophilic moiety Y is capable of anchoring the synthetic fullerene molecule to a lipid membrane.

In another embodiment, the amphiphilic or lipophilic synthetically modified fullerene molecule has the formula Z(C₇₀)Y wherein: Y is a lipophilic moiety covalently connected to Cₚ, optionally through a linking group, at or near a pole thereof, and wherein Z is a hydrophilic moiety covalently connected to Cₚ, optionally through a linking group, at or near a pole opposite to said Y; and, wherein said lipophilic moiety Y is capable of anchoring the synthetic fullerene molecule to a lipid membrane.

In another embodiment the amphiphilic or lipophilic synthetically modified fullerene molecule can have the formula Zₘ-F-Y, wherein:

F is a fullerene of formula Cₚ having p=60-200 carbons, preferably p=60 or 70;

m=1-5 such that each Z is a group AᵣBₛ in which r=1-4, s=1-4, and A is one or more hydrophilic or charged group bonded to the fullerene through one or more linker B;

n=1-5 and each Y is a group DₜEᵥ in which t=1-4, v=1-4 and E is one or more lipophilic group bonded to the fullerene through one or more linker D; and,

X and Y are positioned at or near opposite poles of F.

In certain embodiments the amphiphilic or lipophilic synthetically modified fullerene has a geometrical configuration capable of causing the fullerene molecule to locate within phospholipid bilayers of a cell such that a radical scavenging zone near the equatorial band of the fullerene is situated within or in close proximity to the phospholipid bilayer.

A plurality of such synthetically modified fullerene molecules can be uniformly dispersed in phospholipids, such as in liposomes. The amphipathic fullerene molecules described herein do not generally form vesicles by themselves, but require membrane-forming phospholipids in mole ratios greater than 1:1 (lipid:fullerene adduct) to form vesicles.

The methods described herein also encompass hydrophilic or amphiphilic synthetically modified fullerenes of the formula

Z′ₘ-F-Y′ₙ;

wherein F is a fullerene of formula Cₚ or X@Cₚ, the fullerene having two opposing poles and an equatorial region;

Cₚ represents a fullerene cage having p carbon atoms, and X@Cₚ represents such a fullerene cage having a chemical group X within the cage;

Z′ and Y′ are positioned near respective opposite poles of Cₚ;

m=1-5 and Z′ is a hydrophilic, lipophilic, or amphiphilic chemical moiety;

n=1-5 and Y′ is a hydrophilic or amphiphilic chemical moiety;

p=60-200 and p is an even number; and

X, if present, represents one or more metal atoms within the fullerene (F), optionally in the form of a trinitride of formula Gᵢ₌₁₋₃Hₖ₌₃₋ᵢN in which G and H are metal atoms.

In exemplary variations p is an even number between 60 and 120, with p=60-96 being more common and p=60 or p=70 being preferred. The fullerene can be arranged wherein each chemical moiety Z′ is composed of formula A′ᵣB in which A′ is a hydrophilic, lipophilic or amphiphilic chemical moiety, r=1-4, and B is a chemical linker connecting said A′ to the fullerene, and each chemical moiety Y′ is composed of formula DE′ᵥ in which E′ is a hydrophilic or amphiphilic chemical moiety and, v=1-4, and D is a chemical linker connecting the chemical moiety Y′ to the fullerene.

In another embodiment, the hydrophilic or amphiphilic synthetically modified fullerene molecule has the formula Z′(Cₚ)Y′ wherein: p=60-200 carbons, preferably p=60 or 70; Y′ is a hydrophilic or amphiphilic moiety covalently connected to Cₚ, optionally through a linking group, at or near a pole thereof, and wherein Z′ is a hydrophilic or amphiphilic moiety covalently connected to Cₚ, optionally through a linking group, at or near a pole opposite to said Y′.

In exemplary embodiments, Z′ and Y′ are both amphiphilic; Z′ and Y′ are both hydrophilic; or one of Z′ and Y′ is amphiphilic while the other is hydrophilic. In other embodiments, Z′ is lipophilic and Y′ is hydrophilic or amphiphilic.

In another embodiment, the hydrophilic or amphiphilic synthetically modified fullerene molecule has the formula Z′(C₇₀)Y; wherein Y′ is a hydrophilic or amphiphilic moiety covalently connected to C₇₀, optionally through a linking group, at or near a pole thereof, and wherein Z′ is a hydrophilic or amphiphilic moiety covalently connected to C₇₀, optionally through a linking group, at or near a pole opposite to said Y′.

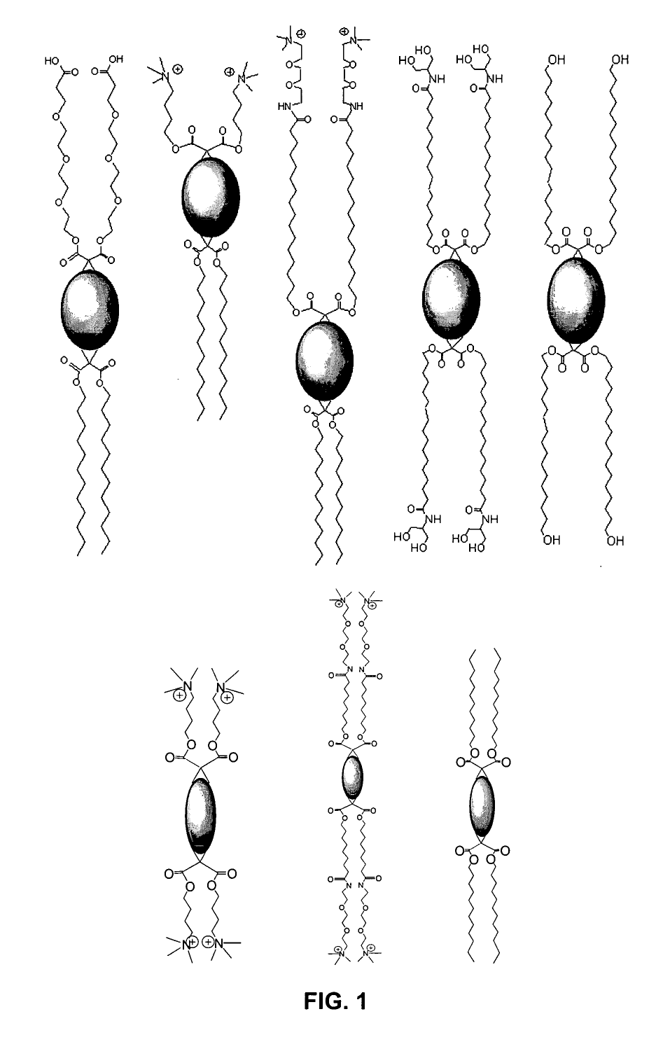

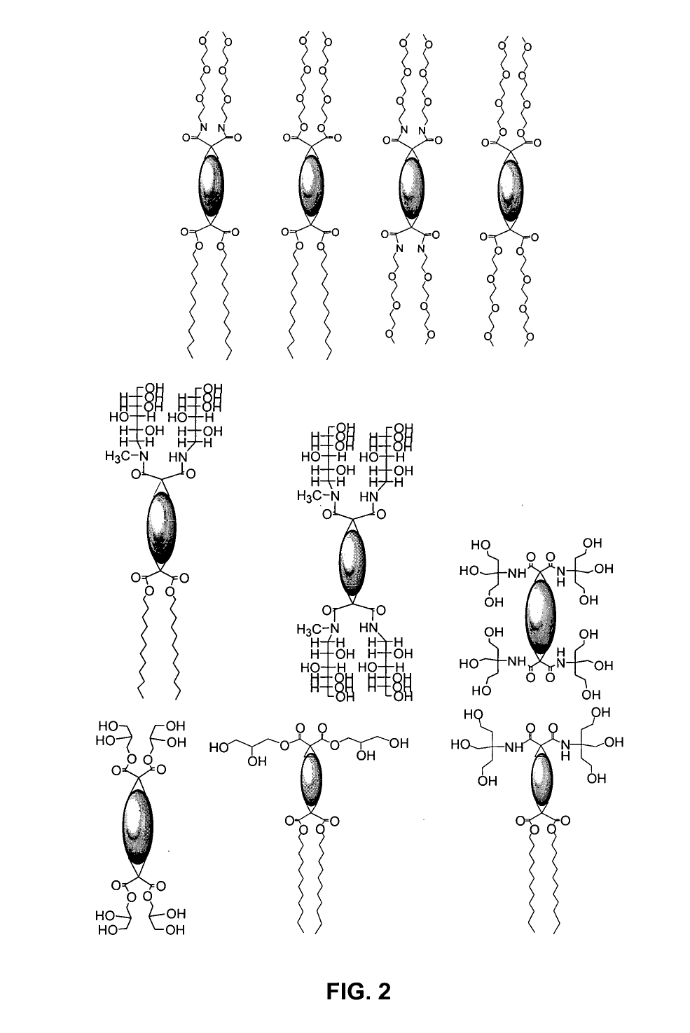

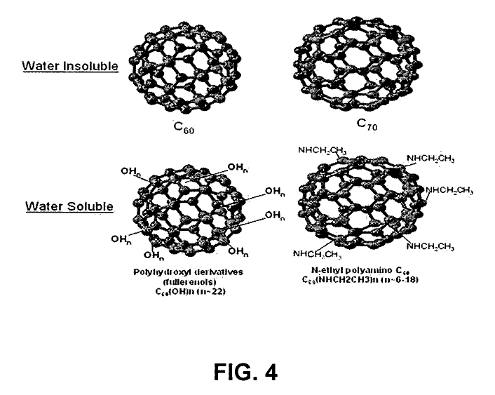

In certain embodiments, the fullerene comprises any one or more of the fullerenes set forth in the present figures. In one embodiment, the fullerene is a fullerene of compound 5 (illustrated in FIGS. 1, 2, and 4). In the present examples, compound 5 comprises C₇₀.

Suitable fullerenes are also described in the following co-pending PCT applications filed concurrently herewith: Attorney Docket No. 1034136-000062, entitled “USING FULLERENES TO ENHANCE AND STIMULATE HAIR GROWTH;” Attorney Docket No. 1034136-000063, entitled “METHOD FOR INHIBITING THE BUILD-UP OF ARTERIAL PLAQUE BY ADMINISTERING FULLERENES;” Attorney Docket No. 1034136-000064, entitled “FULLERENE THERAPIES FOR INFLAMMATION;” and Attorney Docket No. 1034136-000066, entitled “METHOD FOR TREATING WOUNDS BY ADMINISTERING FULLERENES;” the entire disclosures of which are incorporated by reference herein, and in U.S. Patent Application Nos. 61/071,756, filed May 15, 2008, entitled “NEW REACTIONS OF FULLERENES” and 12/073,231, filed Mar. 3, 2008, entitled “STEROID DERIVATIVES OF FULLERENES,” the entire disclosures of which are incorporated by reference herein.

“Arterial plaque” or “atherosclerosis” as used herein are interchangeable. In normal circumstances, the build-up of arterial plaque is a protective response to stresses on the endothelium and smooth muscle cells (SMCs) of the wall of the artery. In response to such stresses, atherosclerosis consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. For example, shear stresses are thought to be responsible for the frequent occurrence of atherosclerotic plaques in regions of the circulatory system where turbulent blood flow occurs, such as branch points and irregular structures.

The first observable event in the formation of an atherosclerotic plaque occurs when blood-borne monocytes adhere to the vascular endothelial layer and transmigrate through to the sub-endothelial space. Adjacent endothelial cells at the same time produce oxidized LDL. These oxidized LDL's are then absorbed in large amounts by the monocytes through scavenger receptors expressed on their surfaces. In contrast to the regulated pathway by which native LDL (nLDL) is absorbed by nLDL specific receptors, the scavenger pathway of uptake is not regulated by the monocytes. Oxidation of LDL into oxidized LDL results in the loss of the recognition of the apo B component by cellular LDL receptors, and in the preferential uptake of oxidized LDL by macrophage “scavenger” receptors. The enhanced endocytosis of oxidized LDL by vascular wall macrophages transforms them into lipid-laden foam cells that characterize early atherosclerotic lesions.

The lipid-filled monocytes are called foam cells, and are the major constituent of the fatty streak. Interactions between foam cells and the endothelial and SMCs which surround them lead to a state of chronic local inflammation which can eventually lead to smooth muscle cell proliferation and migration, and the formation of a fibrous plaque. Such plaques occlude the blood vessel concerned and thus restrict the flow of blood, resulting in ischemia.

Ischemia is a condition characterized by a lack of oxygen supply in tissues of organs due to inadequate perfusion. Such inadequate perfusion can have number of natural causes, including atherosclerotic or restenotic lesions, anemia, or stroke, to name a few. Many medical interventions, such as the interruption of the flow of blood during bypass surgery, for example, also lead to ischemia. In addition to sometimes being caused by diseased cardiovascular tissue, ischemia may sometimes affect cardiovascular tissue, such as in ischemic heart disease. Ischemia may occur in any organ, however, that is suffering a lack of oxygen supply.

The most common cause of ischemia in the heart is atherosclerotic disease of epicardial coronary arteries. By reducing the lumen of these vessels, atherosclerosis causes an absolute decrease in myocardial perfusion in the basal state or limits appropriate increases in perfusion when the demand for flow is augmented. Coronary blood flow can also be limited by arterial thrombi, spasm, and, rarely, coronary emboli, as well as by ostial narrowing due to luetic aortitis. Congenital abnormalities, such as anomalous origin of the left anterior descending coronary artery from the pulmonary artery, may cause myocardial ischemia and infarction in infancy, but this cause is very rare in adults. Myocardial ischemia can also occur if myocardial oxygen demands are abnormally increased, as in severe ventricular hypertrophy due to hypertension or aortic stenosis. The latter can be present with angina that is indistinguishable from that caused by coronary atherosclerosis. A reduction in the oxygen-carrying capacity of the blood, as in extremely severe anemia or in the presence of carboxy-hemoglobin, is a rare cause of myocardial ischemia. Not infrequently, two or more causes of ischemia will coexist, such as an increase in oxygen demand due to left ventricular hypertrophy and a reduction in oxygen supply secondary to coronary atherosclerosis. See, for example, U.S. Pat. No. 6,492,126 for additional information regarding atherosclerosis and ischemia.

Free radical “scavengers” such as vitamins A, E, C, and selenium are believed to react with oxidized LDLs and render them incapable of oxidation. The inhibitory action of these antioxidants thus inhibits the formation of oxidized LDL, thereby lowering the levels of arterial plaque deposits in blood vessels. See, for example, U.S. Pat. No. 6,326,031 for additional background regarding LDL, O-LDL, HDL, and arterial plaque.

Fullerenes effectively block the immune cascade that follows subcutaneous injection of phorbol myristate (PMA). Without wishing to be bound by theory, it is believed that a mechanism of action of this blockade may involve free radical scavenging. Membrane trafficking and permeability may be contributing to the biological response.

Peripheral blood monocytes, when placed in a tissue culture dish will adhere and become macrophages. However they do not normally ingest LDL added to the culture medium. Chemical modification of LDL, e.g., by oxidation, will stimulate macrophages to take up LDL. Another technique for stimulating human peripheral monocytes to ingest LDL is to incubate the macrophages with PMA, as shown by Kruth et al., J Biol Chem, 277:34573 (2002).

Without wishing to be bound by theory, it is believed that a compound that blocks PMA inflammatory response in skin could also block the same pathway in foam cells. Thus, a proposed intracellular mechanism for controlling the uptake of LDL is to use fullerenes to block the inflammatory mechanism in foam cells and thereby preventing these cells from accumulating lipids.

The terms “inhibiting”, “treating,” or “treatment,” and the like are used herein to generally mean obtaining a desired pharmacological and physiological effect, and refer to complete elimination as well as to any clinically or quantitatively measurable reduction in the condition for which the subject is being treated. “Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. More specifically, the fullerenes described herein are used to inhibit the build-up of arterial plaque in a subject. These fullerenes are provided in a therapeutically effective amount to: prevent the disorder (i.e., inhibit the onset or occurrence of the disorder and/or cause the clinical symptoms of the disorder not to develop in a mammal that may be exposed to or predisposed to the disorder but does not yet experience or display symptoms of the disorder); inhibit the disorder (i.e., arrest or reduce the development of the disorder or its clinical symptoms); or relieve the disorder (i.e., cause regression of the disorder or its clinical symptoms). Subjects in need of treatment include all subjects in whom the inhibition of the build-up of arterial plaque is desired.

A “subject in need thereof” refers to any subject or individual who could benefit from the method of treatment described herein. In certain embodiments, a subject in need thereof is a subject in whom the inhibition of the build-up of arterial plaque is desired. The “subject in need thereof” refers to a vertebrate, preferably a mammal. Mammals include, but are not limited to, humans, other primates, rodents (i.e., mice, rats, and hamsters), farm animals, sport animals and pets. In one embodiment, the subject is a mammal such as a human. In certain embodiments, the methods find use in experimental animals, in veterinary application, and/or in the development of animal models for disease.

As used herein, the term “administering” or “introducing” fullerenes to a subject means providing the fullerenes to a subject. Methods of administering fullerenes to subjects are well known to those of ordinary skill in the art and include, but are not limited to, oral, intravenous, intramuscular, parenteral, or local administration. Modes of administration can also include delivery via a controlled release and/or controlled release drug delivery formulation and/or device.

“Sustained release” refers to release of a drug or an active metabolite thereof into the systemic circulation over a prolonged period of time relative to that achieved by oral administration of a conventional formulation of the drug.

“Controlled release” is a zero order release; that is, the drug releases over time irrespective of concentration. Single, multiple, continuous or intermittent administration can be effected.

“Orally delivered drugs” refer to drugs which are administered to an animal in an oral form, preferably, in a pharmaceutically acceptable diluent. Oral delivery includes ingestion of the drug as well as oral gavage of the drug.

“Therapeutic or prophylactic blood concentrations” refers to systemic exposure to a sufficient concentration of a drug or an active metabolite thereof over a sufficient period of time to effect disease therapy or to prevent the onset or reduce the severity of a disease in the treated animal.

“Optional” or “optionally” means that the subsequently described event or circumstance may, but need not, occur, and that the description includes instances where the event or circumstance occurs and instances in which it does not.

“Pharmaceutically acceptable salt” refers to pharmaceutically acceptable salts of fullerenes which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, “pharmaceutically acceptable” means acceptable for use in the pharmaceutical and veterinary arts, compatible with other ingredients of the formulation, and not toxic or otherwise unacceptable commensurate with a reasonable benefit/risk ratio.

A “pharmaceutically acceptable carrier” or “diluent” includes any and all solvents, dispersion media, coatings, antibacterial and anti-fungal agents, isotonic and absorption delaying agents, and the like, compatible with pharmaceutical administration of a composition comprising ferritin-iron complexes. Examples of such carriers or diluents include, but are not limited to, water, saline, Ringer's solutions and dextrose solution. The volume of a pharmaceutical composition or formulation comprising fullerenes is based on the intended mode of administration and the safe volume for the individual patient, as determined by a medical professional.

The selection of carrier also depends on the intended mode of administration. Fullerenes of the present invention may be administered by any of a number of convenient means including, but not limited to systemic administration (e.g., intravenous injection, intraparenteral injection, inhalation, transdermal delivery, oral delivery, nasal delivery, rectal delivery, etc.) and/or local administration (e.g., direct injection into a target tissue, delivery into a tissue via cannula, delivery into a target tissue by implantation of a time-release material, or delivery through the skin via a topical composition such as a cream, lotion, or the like), delivery into a tissue by a pump, etc., orally, parenterally, intraosseously, in the cerebrospinal fluid, or the like. Further modes of administration include buccal, sublingual, vaginal, subcutaneous, intramuscular, or intradermal administration.

In some embodiments, a pharmaceutical composition or formulation comprising plaque targeted fullerenes is administered orally to a subject in whom the inhibition of the build-up of arterial plaque is desired. These fullerenes are substantially absorbed in the intestine and become incorporated into LDL particles in the liver such that a therapeutically effective amount of fullerenes is delivered to the foam cells and the fullerenes block further accumulation of LDL into plaque. As used herein, “pharmaceutical composition” and “pharmaceutical formulation” are interchangeable. In another embodiment, a composition comprising fullerenes is injected directly into the vasculature of a subject in whom the inhibition of the build-up of arterial plaque is desired, such that a therapeutically effective amount of fullerenes are absorbed by arterial plaque to block further accumulation of LDL into arterial plaque. In yet another embodiment, a composition comprising cholesterol modified fullerenes is administered directly to vasculature wherein such cholesterol modified fullerenes form micelles which partition into LDL particles within the vasculature such that a therapeutically effective amount of the fullerenes is absorbed by arterial plaque to block further accumulation of LDL into arterial plaque. Targeting of foam cells in arterial plaque is accomplished through the attachment of groups (i.e., cholesterol derivatives) which home to cholesterol receptors on the foam cells.

A “therapeutically effective amount” or “pharmaceutically effective amount” means the amount of a fullerene that, when administered to a subject in whom the inhibition of the build-up of arterial plaque is desired. Thus a “therapeutically effective amount” is an amount indicated for treatment while not exceeding an amount which may cause significant adverse effects. The “therapeutically effective amount” will vary depending on the types of fullerenes to be administered, the degree of inhibition of the build-up arterial plaque desired, and the age, weight, etc., of the subject to be treated. Methods for evaluating the effectiveness of therapeutic treatments are known to those of skill in the art.

Doses to be administered are variable according to the treatment period, frequency of administration, the host, and the nature and severity of the disorder. The dose can be determined by one of skill in the art without an undue amount of experimentation. The fullerenes are administered in dosage concentrations sufficient to ensure the release of a sufficient dosage unit into the patient to provide the desired level of inhibition of the build-up of arterial plaque. The actual dosage administered will be determined by physical and physiological factors such as age, body weight, severity of condition, and/or clinical history of the patient. In some embodiments, the fullerenes may be administered to achieve in vivo plasma concentrations of the fullerenes of from about 0.01 to 10,000 ng/cc. For example, the methods described in this disclosure may use compositions to provide from about 0.01 to about 100 mg/kg or from about 0.01 to about 10 mg/kg body weight/day of the fullerenes, such as about 30 mg/kg body weight/day of the fullerenes. It will be understood, the ranges provided may also be suitable in the treatment of a given disorder. A practical dosage regimen is a schedule of drug administration that is practical for a patient to comply with. For human patients, a practical dosage regimen for an orally administered drug is likely to be an aggregate dose of less than 10 g/day.

The fullerenes may be in any form suitable for administration. Such administrable forms include tablets, buffered tablets, pills, capsules, enteric-coated capsules, dragees, cachets, powders, granules, aerosols, liposomes, suppositories, creams, lotions, ointments, skin patches, parenterals, lozenges, oral liquids such as suspensions, solutions and emulsions (oil-in-water or water-in-oil), ophthalmic liquids and injectable liquids, or sustained-release forms thereof. The desired dose may be provided in several increments at regular intervals throughout the day, by continuous infusion, or by sustained release formulations, or may be presented as a bolus, electuary or paste.

In various embodiments, a pharmaceutical composition or formulation comprising the fullerenes is prepared by admixture with one or more pharmaceutically acceptable carriers and/or excipients. Other additives and/or active ingredients may be added, if desired, to maximize the preservation of the fullerenes, to optimize a particular method of delivery, or to optimize inhibition of the build-up of arterial plaque in the subject in need thereof. In addition, according to other embodiments, the pharmaceutical composition or formulation comprising fullerenes may include other compositions comprising fullerenes as described herein in combination with other agents suitable for the inhibition of the build-up of arterial plaque.

The fullerenes may be formulated into a variety of compositions (i.e., formulations or preparations). These compositions may comprise any component that is suitable for the intended purpose, such as conventional physiologically acceptable delivery vehicles, diluents and excipients including isotonising agents, pH regulators, solvents, solubilizers, dyes, gelling agents and thickeners and buffers and combinations thereof. Pharmaceutical formulations suitable for use with the instant fullerenes can be found, for instance, in Remington's Pharmaceutical Sciences. Physiologically acceptable carriers are carriers that are nontoxic at the dosages and concentrations employed. Pharmaceutical formulations herein comprise pharmaceutical excipients or carriers capable of directing the fullerenes to the area where the subject in need thereof is a subject in whom the inhibition of the build-up of arterial plaque is desired. Suitable excipients for use with fullerenes include water, saline, dextrose, glycerol and the like.

In various embodiments, the fullerenes are administered to a subject in need thereof in the form of pharmaceutical compositions or formulations. These pharmaceutical compositions or formulations comprise fullerenes and can also include one or more pharmaceutically acceptable carriers or excipients. The excipient is typically one suitable for administration to human subjects or other mammals. In making the compositions of this disclosure, the active ingredient (i.e., fullerenes) is usually mixed with an excipient, and/or diluted by an excipient. When the excipient serves as a diluent, it can be a solid, semi-solid, or liquid material, which acts as a vehicle, carrier or medium for the active ingredient. For additional information regarding suitable methods and formulations for use in the present disclosure are found in REMINGTON'S PHARMACEUTICAL SCIENCES, Mace Publishing Company, Philadelphia, Pa., 17th ed. (1985).

According to one embodiment, the fullerenes may be administered alone, or in combination with any other medicament. Thus, the formulation may comprise fullerenes in combination with another active ingredient, such as a drug, in the same formulation. When administered in combination, the fullerenes may be administered in the same formulation as other compounds as shown, or in a separate formulation. When administered in combination, the fullerenes may be administered prior to, following, or concurrently with the other compounds and/or compositions.

In certain embodiments the pharmaceutical compositions or formulations described herein have a viscosity at 20° C. of from about 5 cps to about 50000 cps, such as from about 500 cps to about 40000 cps, or about 5000 cps to about 30000 cps.

Preparation of Dry Formulations that are Reconstituted Immediately before use also is contemplated. The preparation of dry or lyophilized formulations can be effected in a known manner, conveniently from the solutions of the invention. The dry formulations of this invention are also storable. By conventional techniques, a solution can be evaporated to dryness under mild conditions, especially after the addition of solvents for azeotropic removal of water, typically a mixture of toluene and ethanol. The residue is thereafter conveniently dried, e.g., for some hours in a drying oven.

The method herein is targeted to inhibiting the build-up of arterial plaque in a subject in need thereof. The fullerene-containing preparations described above may be administered systemically or locally and may be used alone or as components of mixtures. In one embodiment the administration is local. The route of administration for the fullerenes may be intravenous, oral, or by use of an implant.

Additional routes of administration are subcutaneous, intramuscular, or intraperitoneal injections of the fullerenes in conventional or convenient forms.

Generally, the pharmaceutical compositions or formulations described herein can be administered as a pharmaceutical or nutritional formulation. These compositions or formulations can be administered orally, intravenously, or as a suppository.

The present disclosure relates to use of any one or more of the fullerenes described herein for inhibiting the build-up of arterial plaque. The present disclosure also relates to use of any one or more of the fullerenes described herein for manufacturing a medicament, particularly a medicament for inhibiting the build-up of arterial plaque.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed. All publications, patents, patent applications and other references cited herein are hereby incorporated by reference.

While the invention disclosure has been described in detail with reference to certain embodiments thereof, it will be apparent to one skilled in the art that various changes can be made, and equivalents employed, without departing from the scope of the invention.disclosure. In addition, the following examples are illustrative of the methods described herein and should not be considered as limiting the foregoing disclosure in any way.

### EXAMPLES

### Example 1

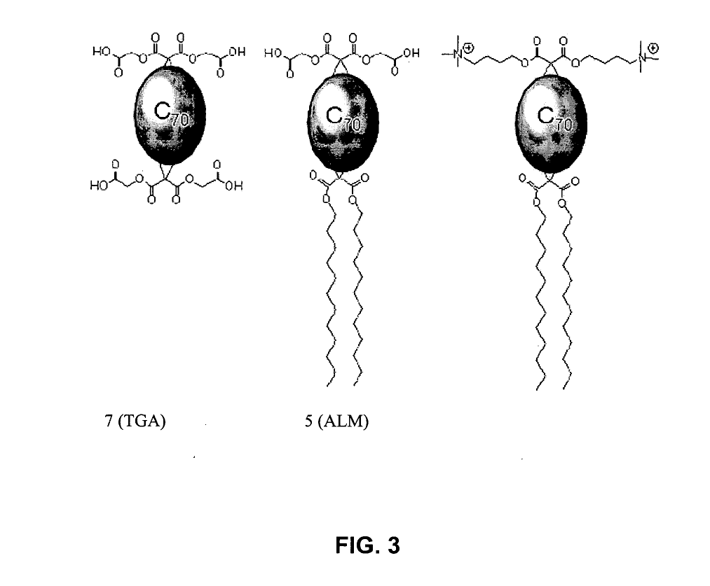

Cultured monocytes will adhere to tissue culture flasks and differentiate into macrophages. Such cultured macrophages can be studied as a model system for foam cells. Human peripheral blood monocytes were transferred to plastic tissue culture dishes. One dish was treated with fullerene compound 5 (illustrated in FIG. 3) formulated in liposomes at concentrations ranging from 0.01 ng/ml to 50 ng/ml. Another dish was treated with empty liposomes containing no fullerene compound. Shortly thereafter, phorbol myristic acetate was added at a concentration of 1 μg/ml to the culture medium of both groups. Shortly thereafter LDL was added to both groups at a concentration of 2 mg/ml. After 24 hours the cells were examined under a microscope. The cells treated with empty liposomes appeared vacuolated, similar to foam cells, while those treated with fullerene compound 5 had few vacuoles. This suggests that fullerene compound 5 blocked the PMA induced uptake of LDL.

### Example 2

The U937 cell line is established from a diffuse histiocytic lymphoma and serves as an in vitro model for monocyte to macrophage differentiation. To induce foam cell formation, U937 monocytic cells were seeded at 1×10⁶ cells/mL in 24 well plates prior to experimental treatments. Low density Lipoprotein (LDL) from human plasma was dialyzed against PBS for 24 hours at 4° C. to remove EDTA. The EDTA-free LDL was then oxidized by incubation in CuSO₄ at 10 umol/L at 37° C. for 12 hours and then dialyzed in PBS containing EDTA 0.1 mmol at 4° C. for 24 hours. For differentiation into macrophage cells, the U937 cells were treated with 0.7 ug/mL phorbol-myristic acid (PMA) and incubated for 24 hours at 37° C., 6% CO₂. Oxidized-LDL (10 ug/mL) was added to the PMA-differentiated macrophage cells and incubated for 48 hours at 37° C., 6% CO₂. To test the effects fullerenes have on differentiation a portion of cells were incubated with various concentrations of fullerene derivatives 5 (ALM) and 7 (TGA) (2.5 ug/mL, 5.0 ug/mL, and 25 ug/mL) for 24 hours at 37° C., 6% CO₂. The viability of the differentiated U937 macrophage cells was determined by trypan blue exclusion.

Differentiated foam cells treated with fullerene derivatives have different responses to lipids provided in culture. Upon analyzing lipid uptake in cells incubated with and without fullerene derivatives, Oil Red 0 staining showed a significant amount of lipid accumulation in the cytoplasms of cells incubated with PMA and various concentrations of Ox-LDL, indicating foam cell transformation. Untreated U937 monocytes showed very little staining. However, cells pre-incubated with 5 (ALM) prior to the addition of PMA and Ox-LDL had significantly less staining than those not receiving 5 (ALM), indicating less lipid accumulation. (Data not Shown.)

While various embodiments have been particularly shown and described herein, it will be understood by those skilled in the art that various changes in form and details may be made therein without departing from the spirit and scope of these embodiments as further defined by the appended claims.

1. A method of inhibiting the build-up of arterial plaque, comprising:
administering a therapeutically effective amount of one or more fullerenes to a subject in need thereof.

2. The method of claim 1, wherein said fullerenes inhibit accumulation of LDL in foam cells of the subject.

3. The method of claim 1, wherein said fullerenes are delivered directly to the foam cells of the subject.

4. The method of claim 1, wherein said fullerenes are administered as cholesterol modified fullerenes.

5. The method of claim 1, wherein said fullerenes are administered orally or intravenously.

6. The method of claim 1, wherein said fullerenes are administered as a pharmaceutical composition which comprises at least one carrier and/or at least one excipient.

7. The method of claim 1, wherein said fullerenes are administered to the subject in combination with at least one other active ingredient.

8. The method of claim 1, wherein said subject is a human.

9. The method of claim 1, wherein at least one of said one or more fullerenes is a synthetically modified fullerene of the formula
Zₘ-F-Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms.

10. The method of claim 9, wherein p is 60 or 70.

11. The method of claim 10, wherein p is 70.

12. The method of claim 1, wherein at least one of said one or more fullerenes is a synthetically modified fullerene of the formula
Z(Cₚ)Y
wherein p=60-200 carbons, preferably p=60 or 70; Y is a lipophilic moiety covalently connected to Cₚ, optionally through a linking group, at or near a pole thereof, and wherein Z is a lipophilic moiety, amphiphilic moiety, or a hydrophilic moiety covalently connected to Cₚ, optionally through a linking group, at or near a pole opposite to said Y.

13. The method of claim 12, wherein Cₚ is C₇₀.

14. The method of claim 1, wherein at least one of said one or more fullerenes is a synthetically modified fullerene of the formula
Z′ₘ-F-Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms.

15. The method of claim 14, wherein p is 60 or 70.

16. The method of claim 15, wherein p is 70.

17. The method of claim 1, wherein at least one of said one or more fullerenes is a synthetically modified fullerene of the formula
Z′(Cₚ)Y′
wherein: p=60-200 carbons, preferably p=60 or 70; Y′ is a hydrophilic or amphiphilic moiety covalently connected to Cₚ, optionally through a linking group, at or near a pole thereof, and wherein Z′ is a hydrophilic or amphiphilic moiety covalently connected to Cₚ, optionally through a linking group, at or near a pole opposite to said Y′.

18. The method of claim 17, wherein Cₚ=C₇₀.

19. The method of claim 1, wherein at least one of said one or more fullerenes is a compound shown in the present figures.

20. The method of claim 1, wherein at least one of said one or more fullerenes is selected from the group consisting of compound 5, compound 7, and combinations thereof.

21. A synthetically modified fullerene of the formula
Zₘ-F-Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms,
for use in inhibiting the build-up of arterial plaque.

22. A synthetically modified fullerene of the formula
Zₘ-F-Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms,
for preparation of a medicament for inhibiting the build-up of arterial plaque.

23. A synthetically modified fullerene of the formula
Z′ₘ-F-Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms,
for use in inhibiting the build-up of arterial plaque.

24. A synthetically modified fullerene of the formula
Z′ₘ-F-Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms,
for preparation of a medicament for inhibiting the build-up of arterial plaque.
